(19) United States
(12) Patent Application Publication
(10) Pub. No.: US 2006/0104952 A1
(43) Pub. Date: May 18, 2006

(54) Genomic barrier to viral disease

(76) Inventor: Fredric D. Abramson, (US)

Correspondence Address: Bell, Boyd & Lloyd LLC, P.O. Box 1135, Chicago, IL, 60690-1135, US

(21) Appl. No.: 11/124,312

(22) Filed: May 6, 2005

Related U.S. Application Data
(63) Continuation of application No. 10/912,246, filed on Aug. 6, 2004.

(51) Int. Cl. C12Q 1/68; C12N 15/87; A61K 48/00

(52) U.S. Cl. 424/93.2; 435/6; 435/455

(57) ABSTRACT

The present disclosure describes methods for the formation of a genomic barrier to viral infection as a means preventing or mediating viral disease. The formation of a genomic barrier would complement a person's immune system response based on the ability of a specific person to create a genomic environment which inhibits cellular exploitation by invading virions, which is normally dependent on the person's genotype and gene expression patterns (Expressitype). However, by modulating gene expression patterns using various agents, including foodstuffs, extracts and/or pharmaceutical supplements, individuals with essentially the same genotypes (relative to the invading virus) may be treated by the methods described to produce such a barrier. By using microarrays and other related technologies such Expressitypes and compounds which re-regulate gene expression to establish genomic barriers can be identified and carried out, respectively, with the methods as described.

This application claims benefit of U.S. Provisional Application No. 60/492,316, filed Aug. 6, 2003, the entire contents of which is incorporated herein by reference.

### BACKGROUND OF THE INVENTION

1. Field of the Invention

The invention relates generally to regulation of gene expression and methods of treating diseases, more specifically, to tactical modulation of host gene expression to prevent and mediate viral disease by creating a gene landscape in the host such that invading virions cannot exploit host genetic machinery.

2. Background Information

An invading virus usually high-jacks parts of the cells machinery in order that the virus can reproduce. In systems biology terms, an invading virus redirects parts of the host's genomic system. This new subsystem activity results in successful viral replication. Both the redirected subsystem and the viral replication disrupt the host's normal genomic system, with the consequence of illness and even death.

Many viruses, though not all, use the host's genetic machinery to replicate, further, the spread of a virus in humans is not uniform. Different population groups and age groups have different incidence rates and susceptibilities. Moreover, these population groups differ in both their genetic composition as well as in their dietary consumption habits.

A major part of the difference in susceptibility and severity has a lot to do with the host's immune system. However, the immune system variation by itself does not explain everything. Alternatively, there may be genomic barriers that prevent a virus from replicating in one host which are absent in another host where the virus flourishes. Furthermore, since gene expression reflects actual genetic activity, a potential genomic barrier to infection may reflect differences in gene expression, not just genotype, gene expression being typically measured by the number of copies of messenger RNA produced by the gene. Therefore, given two people with the same genes that an invading virus uses to replicate, the gene expression pattern in one may block infection while the gene expression pattern in the other essentially invites the virus in.

The potential use of weaponized viruses as bioweapons is well known. Conventional research approaches include creating a vaccine or an anti-viral drug. However, these have documented drawbacks, including the ability to find an effective, timely solution. For example, there is not yet a vaccine for West Nile Virus, though its known existence dates back six decades, and according to WHO, a SARS vaccine is at least two years away.

The invention as envisaged focuses on a method to avoid the initial genomic disruption. This is quite complementary with other research directed at destroying the virus either through the host immune system or through anti-viral drugs. The present invention is based on studies and observations from epidemiology, genomics, and nutrition. Because certain viruses must gain access to the cell's machinery, it is believed that it will be possible to block that access by means of dietary adjustments.

Thus, a need exists for preventing viral infection by denying such infectious agents the ability to exploit the genome activities of the host. The present invention satisfies this need and provides additional advantages.

### SUMMARY OF THE INVENTION

The present invention relates to the formation of a genomic barrier to viral infection as a means of preventing or mediating viral disease.

In one embodiment, a method of re-regulating the expression of one or more genes of a host to prevent or mediate viral infection is envisaged, including generating a gene expression pattern for a host, contacting the host with a battery of one or more agents, and determining the resulting gene expression pattern of the host, where the determining step includes relating the pattern of gene expression resulting from agent re-regulation with an inability of the virus to establish infection.

In a related aspect, the resulting pattern resulting from such a method is indicative of a host whose gene expression environment makes unavailable the cellular machinery necessary to establish a viral infection, where the cellular machinery includes cell surface molecules, nucleic acids, cytoplasmic molecules, or transcription factors.

In a further related aspect, the agent up- and/or down-regulates the expression of cell surface molecules or transcriptional factors. Moreover, such agents modulate one or more host genes by up and/or down regulation of such genes.

In another related aspect, agents are selected from foodstuffs, extracts from foodstuffs, or dietary supplements. Further, such methods can be carried out in cell culture or with whole organisms. In a related aspect, the agent is administered in the form of foodstuffs, extracts from foodstuffs, or dietary supplements.

In another embodiment, a method of preventing or mediating viral infection in a subject in need thereof including administering an agent which re-regulates the gene expression profile of a subject, where the resulting gene expression profile of the subject correlates with an inability to support the cellular machinery necessary to establish a viral infection.

In one embodiment, a method of identifying agents which prevent or mediate viral infection by re-regulating the gene expression pattern of a host is envisaged, including generating a gene expression pattern for a host, contacting the host with at least one or more viruses, determining the gene expression pattern of the host, contacting the host with a battery of one or more agents, and identifying the one or more agents which re-regulate the gene expression pattern of the host, where the identifying step includes determining that the viruses are unable to establish an infection subsequent to contacting the host with the agents.

In another embodiment, a method for targeting one or more common genes that prevent infection from a plurality of viral strains is disclosed, including generating viral Expressitype Signature (VES) patterns for a plurality of non-susceptible hosts, where the hosts have been exposed to various strains of viruses, comparing the patterns of the hosts, identifying similar expression profiles for genes within the VES patterns, and generating similarity profiles, where similar expression profiles between genes are associated with host products that when expressed commonly prevent or mediate infection by the virus strains.

In one embodiment, a method of identifying at least one Viral Expressitype Signature (VES) of a host susceptible to infection by at least one infectious agent is envisaged, including determining the pattern of gene expression upon infection of the susceptible host by an agent, comparing the pattern obtained with at least one gene expression pattern of the susceptible host existing prior to infection by the viral agent, comparing the pattern of the susceptible host with a gene expression pattern of an non-susceptible host exposed to the same agent, comparing the patterns and determining the differences between the patterns, where the determining step resolves the pattern of susceptible host gene expression that is associated with infection via the infectious agent, thereby identifying the VES of the susceptible host. In a related aspect, the determining step may include identifying genes whose concentration does not change.

In another related aspect, the VES is designated as a variable VES or a constant VES, where the variable VES contains genes that are associated with a particular virus strain. On the other hand, a constant VES contains genes that are associated with a plurality of viral strains. In a further related aspect the identified VES is diagnostic for susceptibility to infection by an infectious agent.

In another embodiment, a method of identifying at least one Viral Expressitype Signature (VES) of a host which is not susceptible to infection when exposed to at least one infectious agent is envisaged, including determining the pattern of gene expression upon exposure of a non-susceptible host to an agent, comparing this pattern with the gene expression pattern of the non-susceptible host extant prior to exposure to the agent, comparing the non-susceptible pattern with a gene expression pattern of an infected susceptible host, comparing the pattern with at least one gene expression pattern of a susceptible host extant prior to infection and determining the differences between the patterns, where the determining of step resolves the pattern of non-susceptible host gene expression that is associated with exposure to the infectious agent, thereby identifying the VES of the non-susceptible host.

In another embodiment, a method of modulating the Viral Expressitype Signature (VES) of a host susceptible to a specific viral infection to resemble that of a VES of a non-susceptible host is disclosed, including generating a VES for a susceptible and non-susceptible host, contacting the susceptible host with a battery of one or more agents, determining the VES of the susceptible host, comparing the VES of the susceptible host with the VES of the non-susceptible host, reiterating steps these steps, and determining whether the contacting results in a susceptible host VES that is substantially identical to the VES of the non-susceptible host.

In another embodiment, a method of identifying agents which modulate the gene expression pattern of a host susceptible to a specific viral infection to resemble that of a Viral Expressitype Signature (VES) of a non-susceptible host is envisaged including, generating a VES for a susceptible and non-susceptible host, contacting the susceptible host with a battery of one or more agents, determining the VES of the susceptible host, comparing the VES of the susceptible host with the VES of the non-susceptible host, reiterating steps, and identifying the one or more agents which modulate the gene expression pattern of the susceptible host, where the identifying step includes determining whether the contacting results in a susceptible host VES that is substantially identical to the VES of a non-susceptible host.

### BRIEF DESCRIPTION OF THE DRAWINGS

FIG. 1 is an illustration of human genes (ovals) whose gene expression is influenced by influenza A virus (rectangles). See Table I for gene identity.

FIG. 2 is an illustration of how gene expression of a particular gene from FIG. 1 is controlled by small molecules (ovals) and plays a role in other health conditions (squares). The expression of gene CCL5 may be controlled by hydrocortisone, dexamethasone, and betamethasone. The CCL5 gene affects rapid progress of HIV-1 disease and delayed progression of HIV-1 diseases.

### DETAILED DESCRIPTION OF THE INVENTION

Before the present compositions and methods are described, it is understood that this invention is not limited to the particular methodology, protocols, and reagents described as these may vary. It is also to be understood that the terminology used herein is for the purpose of describing particular embodiments only, and is not intended to limit the scope of the present invention which will be described by the appended claims.

It must be noted that as used herein and in the appended claims, the singular forms “a”, “an”, and “the” include plural reference unless the context clearly dictates otherwise. Thus, for example, reference to “a subject” includes a plurality of such subjects, reference to “an agent” includes one or more agents and equivalents thereof known to those skilled in the art, and so forth.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the methods, devices, and materials are now described. All publications mentioned herein are incorporated herein by reference for the purpose of describing and disclosing the molecules, compounds, and methodologies which are reported in the publications which might be used in connection with the invention. Nothing herein is to be construed as an admission that the invention is not entitled to antedate such disclosure by virtue of prior invention.

As used herein, “micronutrient,” including grammatical variations thereof, means any substance that is ingested into the body through food, dietary supplements or pharmaceuticals that also is bioactive in terms of a direct or indirect effect on the expression of one or more genes.

As used herein, “susceptible host,” including grammatical variations thereof, means cell or multi-cellular organism that is not resistant to infection by a virus.

As used herein, “non-susceptible host,” including grammatical variations thereof, means a cell or multi-cellular organism that is resistant to infection by a virus.

As used herein, “modulated,” including grammatical variations thereof, means to vary the amplitude, frequency, or phase.

As used herein, “substantially identical,” including grammatical variations thereof, means being of such close resemblance as to be essentially the same.

As used herein, “virus,” including grammatical variations thereof, means any of a large group of submicroscopic infective agents that are regarded either as extremely simple microorganisms or as extremely complex molecules, that typically contain a protein coat surrounding an RNA or DNA core of genetic material but no semipermeable membrane, that are capable of growth and multiplication only in living cells, and that cause various important diseases in humans, lower animals, or plants.

As used herein, “cellular machinery,” including grammatical variations thereof, means the organized complex of inorganic and organic substances that comprise a cell.

In a related aspect, cell surface molecules include, but are not limited to, all organized complexes of inorganic and organic substances that comprise the outer boundaries of a cell. For example, a receptor is a cell surface molecule.

In another related aspect, nucleic acids include, but are not limited to, DNA and RNA.

In another related aspect, cytoplasmic molecules include, but are not limited to, all organized complexes of inorganic and organic substances that comprise the internal volume of a cell.

In another related aspect, transcriptional factors include, but are not limited to, any protein required to initiate or regulate the synthesis of either RNA on a template of DNA or DNA on the template of RNA.

As used herein, “genomic barrier,” including grammatical variations thereof, means the genetic material of an organism which prevents a virus from infecting a cell.

The present invention envisages a method of preventing viral disease through direct intervention with the host genome that creates a genomic barrier against the virus. In a related aspect, by tactically up- or down-regulating key genes in the host genome, an invading virus would be unable to highjack the host genetic machinery to replicate. The effect would prevent rapid replication and proliferation of the virus, giving other host defense mechanisms the chance to work effectively. Further, such a genomic barrier to viral infection can be implemented person by person.

Each virus appears to produce its own signature gene expression pattern in the host. This observation includes the work of Dr. Jett and her colleagues at the Walter Reed Army Medical Institute. In the present invention, the term Viral Expressitype Signature (VES) is used to designate a subset of that signature, to designate the identity of the genes in a person's genome that a virus uses and co-opts in order to replicate. The term “Expressitype” helps to distinguish actual genetic activity at a point in time from the identity of the genes themselves, the Genotype.

The present invention envisages that a VES has predictable attributes that justify pursuing the genomic barrier approach.

The first attribute is constancy; i.e., for a given viral strain, the genes that are targeted in the VES will be the same no matter who the host might be. In other words, the virus hunts for the same genes in each person.

One implication of constancy is that a specific target may have one or more alleles in the VES that, because of the nature of that mutation, cannot be used by virus. Thus, with that allele, the person survives infection.

A second attribute is traceability. Traceability means that when the viral expressitype signatures of different strains are compared, most of the target genes will be the same, but there will be some differences. This is similar to the variable and constant portions found in the immunoglobulins.

The implication for traceability is that as a virus mutates and new strains emerge, most of the genes to be grabbed in the host are the same. The changed genes in the VES may reflect the structural differences that render a vaccine against one strain relatively ineffective against the next strain. At the same time, if genes in the constant portions of the VES can be re-regulated, then this potentially provides a more universal preventive solution, one that works against a class of viruses, not just one strain.

A third attribute is survivability. Survivability means that the genes taken over by the virus for its own replication purposes are genes that the host can spare. Which simply means that the host doesn't die, or doesn't die too quickly, when the genes are hijacked.

Survivability suggests that the best genes for a virus to grab are genes that are largely irrelevant to the survival of the host. Good candidates might include genes involved with the growth of nose hair. Another might be a gene that was once important but is no longer used, such as those involved in the eruption of infant teeth. Another candidate class includes genes that will one day be important, but are not now. These genes may be those implicated in processes associated with aging, or something that arises sporadically such as the production of breast milk after pregnancy.

The present invention discloses methods to create a genomic barrier that prevents an invading virus from using the target host's genetic machinery to replicate. By locking-down a person's genome from viral use, an infected person will neither show clinical signs of disease nor be able to spread the virus to others. In a related aspect, such methods will be useful to protect both the military and the general civilian population.

In a further related aspect, an appropriately calibrated chemical mix that is matched to a person's genomic profile can effectively create this genomic barrier, and this mix can be adjusted to accommodate the kind of genetic heterogeneity that is found in every population. Moreover, based on the observed epidemiology of influenza, most components of this mix may be available in the normal dietary stream.

The present invention uses systems biology principles to target specific gene combinations in the host that are implicated in viral replication, and to up or down regulate those genes in such fashion that an invading virus cannot replicate inside the cell. These same systems biology principles are used to identify which factors in a person's dietary environment would be most successful in accomplishing that re-regulation. Such re-regulation based on factors in the dietary environment comprises NutriGenomics intervention.

Such an approach provides a different defensive and preventive approach that complements vaccines, anti-viral drugs, and others being researched. Several distinguishing features are as follows:

1. The class of personalized solutions is likely to successfully block many strains of a given virus. In other words, the specific host genes being re-regulated would create a genomic barrier against different strains. This is analogous to using the constant portions of an immunoglobulin.

2. It is likely to have a positive effect on both military and civilian morale. The threat of a bio-weapon preys on primordial fears of the unknown. Part of this fear relates to not being able to take personal actions to protect oneself: the felling of dread against some outside agency. Solutions such as vaccines do not adequately allay those fears, partly because vaccines are things being done to someone and partly because of suspicions regarding the safety and efficacy of such modalities. One has only to look at the resistance among health workers to smallpox vaccinations, and the general public health failure to have sufficient people immunized against the flu. On the latter, it does not help that the current vaccine doesn't protect against a new flu observed in Texas in late 2003.

3. The genomic barrier approach is likely to work with other types of viral and possibly non-viral pathogens, to the extent these pathogens use the host's genetic machinery as part of its pathogenesis.

4. It is likely to be more easily deployable than vaccines or other existing methods, both in response to a new specific threat activity and in preparation for likely exposure to a known threat.

The present invention incorporates observations of epidemiologic viral disease patterns, combined with research on nutrition-illness and gene-illness relationships as well as published genomics studies relating to specific viruses. For example, it is well known that not every person exposed to a virus becomes ill, and that the severity of illness will vary from one person to the next. In addition to well-documented differential risks associated with age and related health conditions, different infection and illness incidence rates are observed in populations grouped by geography and ethnicity. Other research shows that certain diets can increase resistance. There also appears to be significant genetic factors relating to susceptibility.

These findings are traditionally interpreted to involve differences in the immune system. While the immune system is a major factor in viral disease resistance, other factors such as the state of the target host's genome must also be considered.

In one embodiment, the present invention uses systems biology principles to identify host gene combinations that are involved with the viral infection and that, when expression is changed, create a barrier environment that either prevents or slows viral replication without otherwise jeopardizing the person's health. In a related aspect, the methods can be used to identify combinations of dietary chemicals that, when appropriately delivered to the host, can orchestrate those gene combinations in the proper direction.

The term “dietary chemicals” as used herein, is comprehensive, and includes dietary supplements and pharmaceuticals in addition to food.

Influenza A.

The present method can be tested and validated on any viral agent including, but not limited to, Influenza A.

Influenza A's structure is well characterized, and can be easily engineered using existing moderate level technology.

Influenza A is delivered both by aerosol and by contact. Thus, one delivery mode would be contagious individuals circulating in crowded areas, like airports and malls. This would rapidly create additional infectious vectors with high mobility, meaning rapid and broad spread.

Influenza A is dangerous under normal conditions. Over 40,000 people die each year in the U.S. from complications relating to Influenza. This is roughly the same number of people who die from breast cancer.

Influenza A can become highly lethal. The 1918 Spanish Flu epidemic killed over 600,000 Americans in two months, and as many as 40 million world-wide. Simulating the 1918 epidemic spread against the current U.S. population, over 16 million casualties would result, including 3.5 million deaths.

The economic consequences of a severe influenza illness include the fact that it would easily overwhelm the U.S. medical system. This happened in Los Angeles, for example, in the late ‘50’s in the Asiatic Flu epidemic.

In military settings, delivery of a weaponized influenza A could quickly incapacitate a fighting force and there is no guarantee that the many early threat detection methods being developed would work against influenza given its incubation and infectivity periods.

Influenza A would be tested in stages, using first human cell lines to identify baseline candidate target genes and chemical interventions. Second, without direct intervention, tracking in-vivo in a large human population during an influenza season would be accomplished. This tracking would capture repeat gene expression measures in each participant as well as their routine dietary inputs.

The response challenge to a lethal virus is essentially the same, regardless of whether the virus occurs through natural causes or through a bioterrorist attack. The danger from the National security perspective is illustrated with the influenza virus. Because recent advances in approaches such as the reverse genetic system^{1,2} are now available, it is likely that a lethal influenza A virus can be generated in the laboratory. Geiss points out, as an example, that the pathogenic H5N1 virus has already been generated in at least one laboratory using this reverse genetic system.^{3,4,8}

As there is every reason to believe that similar recombinant DNA techniques can be used make such viruses transmissible, it should be possible to introduce mutations into such a recombinant virus so that it is resistant to currently available influenza antivirals (M2 inhibitors: amantadine and rimantadine; and NA inhibitors: zanamivir and oseltamivir).^{5,6,7}

Influenza virus infection results in up- and down-changes in the expression of multiple genes in the host genome, as part of the virus's infective process.⁸ Observed host gene expression changes may be reflect activity in the host's defense mechanisms, and/or the virus's use of the host genetic machinery to replicate.

The identity of the host genes that are up- or down-regulated appear to be constant for a particular virus, but show differences from one subspecies to another. These common genes may be highly useful intervention targets because their functions may be required by all of the subspecies.

New influenza variants appear as often as annually. Influenza A and influenza B strains appear to have overlaps in the identity of the host genes attacked, while demonstrating significant differences in other genes attacked. The profile of genes up- and down-regulated by a specific virus provides a “Viral Expressitype Signature” that can be used to identify the virus and target preventive intervention.

Nutrients and other dietary ingredients, including dietary supplements, influence gene expression. A particular ingredient (“micronutrient”) can up- or down-regulate many genes. For example, retinoic acid (Vitamin A) appears to directly or indirectly regulate over 530 genes,⁹ coenzyme Q10 influences expression in at least 115 genes,¹⁰ ginkgo biloba extract up-regulates over 40 genes.¹¹ Further, ordinary foods are made up of hundreds of different chemicals, many of which are bioactive. Micronutrients and genes interact as part of a dynamic system that sustains life and health.

Epidemiologic studies show that individual susceptibility to influenza infection and illness varies both within and among populations. Part of the variation can be explained by acquisition of immunity through prior exposure. This is thought to explain why the influenza A virus in the 1918-1919 Spanish Flu pandemic affected mainly healthy young adults, 15 to 35 years old with no underlying disease.¹²

Part of the variation can be explained by the presence or absence of compromising cofactors in the host, e.g., lung, heart or kidney disease, or disease-based immunosuppression.¹³ The presence of cofactors explains some of the mortality associated, for example, with annual influenza epidemics. Part of the variation appears to be related to genetic differences in hosts, where persons of one genotype are more or less susceptible to a viral infection.

Alternatively, the variation may be related to differences in both short and long-term dietary habits, which may include the presence or absence of such items as antioxidants, Omega-3 fatty acids, dietary fat, zinc, and certain vitamins. Selenium deficiency, for example, not only appears to increase the pathology of an influenza virus infection,¹⁴ it seems to stimulate within-host mutations of the replicating influenza virus.¹⁵

Therefore, it is scientifically reasonable to conclude that specific combinations of micronutrients will up- or down-regulate specific genes in a given host, where such change in gene expression effectively prevents a virus from utilizing the host's genetic machinery to replicate.

Influenza is associated with both epidemics and pandemics in humans. In addition, the influenza virus has a unique dependence on the host cell nuclear function to synthesize its own mRNA.¹⁶

Most people consider the flu to be a severe but temporary problem because the duration is short, typically one or two weeks, and because the deaths associated with flu tend to be confined to people who have already compromised immune systems as a result of one or more health problems. But more dangerous threats can occur. The 1918-1919 “Spanish Flu” pandemic, for example, which involved influenza A, killed between 20 and 40 million worldwide.^{8,17,18,19,20,21}

Global pandemics involving particularly virulent strains take place roughly once a decade since the 1930's. In the 1957-1958 pandemic, the attack rate of clinical influenza exceeded 50% of urban populations, and it is believed that additional 25 percent may have been sub-clinically infected. Influenza A epidemics begin abruptly and reach a peak over a 2- to 3-week period. They typically last for 2 to 3 months.²² New influenza A virus strains appear frequently. Even with annual vaccinations in the U.S., between 20,000²³ and 40,000¹³ die each year in the U.S. from influenza virus infection and its complications.

More important for contemporary concerns, influenza A virus has great potential as a bioterrorist weapon.¹³ Influenza A viruses are not only responsible for the widespread human epidemics and pandemics that have caused high mortality rates, they have been isolated from a wide variety of avian and mammalian species.¹²

Re-Regulating Agents and Compounds

Re-regulating agents and compounds identified via assays such as those described herein may be useful, for example, in re-regulating genomic activity of host cells such that genomic barriers are created to prevent or moderate viral infection. Assays for testing the efficacy of compounds identified in the methods disclosed can be tested in animal model systems. Such animal models may be used as test agents for the identification of drugs, pharmaceuticals, therapies and interventions which may be effective in treating viral infections.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to unaffected cells and, thereby, reduce side effects.

The data obtained from cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

Pharmaceutical compositions for use in accordance with the present invention may be formulated in a conventional manner using one or more physiologically acceptable carriers or excipients.

Thus, the compounds and their physiologically acceptable salts and solvates may be formulated for administration or delivery by oral or parenteral administration. Alternatively, such agents may be administered or delivered by foodstuffs and/or foods themselves.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The following examples are intended to illustrate but not limit the invention.

### EXAMPLES

### Example 1 Influenza A

The present method can be tested and validated on Influenza A. The literature and experts in the field indicate that Influenza A is a significant terrorist threat to both military and civilians. In addition, its annual natural appearance makes it a significant general health threat. It is therefore a logical focus to start experiments.

Most new influenza viruses emerge because of antigenic drift, which involves minor mutations on the HA antigens. This is the reason that each new flu season requires a new vaccine to the novel HA subtype that is expressed in the mutated virus. Major antigenic variations arise from antigenic shifts. These resulting viruses may be associated with pandemics such as those described above, and are restricted to influenza A viruses.²⁴

Influenza is transmitted by inhaling droplets from a sneeze or cough from an infected person. The initial site of infection is the upper respiratory tract and virus replication begins in columnar epithelial cells of respiratory epithelium. The virus spreads to neighboring cells and symptoms appear 48 hours post infection. Within 3 days symptoms begin to subside and in about 7 days the immune system contains the infection but complete recovery can take a 2-3 weeks. However, in some individuals, because of a weakened or suppressed immune system or in the case of a very invasive strain of virus, life-threatening complications can ensue.

The influenza viruses are helical, envelope segmented RNA viruses. There are four influenza virus genera, two of which, influenza A and influenza B are the most common causes of human illness.^{25,26} Influenza types can be distinguished by serological differences between nucleocapsid antigens. There are also different strains within the different types of influenza viruses that can be distinguished by serology.

The influenza A virus contains 10 genes among 8 RNA segments. Each gene appears to be encapsulated but is susceptible to degradation by ribonucleases. Each of the smallest two RNA segments yields two separate transcripts by differential splicing. Therefore the influenza A virus has 10 different proteins.

Influenza A viruses are classified based on antigenic reactivity of two envelope proteins, namely hemaglutinin (HA or H) and the neuraminidase (NA or N). This designation is commonly written as HxNy where x and y are numbers HA (1-14) and N (1-9).

Segment 8 encodes an mRNA that is alternatively spliced to express the nonstructural protein-1 (NS1) and the nuclear export protein, NEP.²⁷ The NS1 protein, which binds double-stranded RNA and forms dimers in vivo, has been suggested to perform several important accessory functions for the optimal replication of the virus in its host.¹⁶

The preventive method of the present invention should to act independently of the host cell antiviral response, which suggests that the existence of the NS1 protein may not permit successful virus propagation and survival if the host genetic machinery itself is denied the virus.

The hemaglutinin of influenza virus binds the N-acetylneuraminic acid containing glycoproteins on the surface of erythrocytes resulting in lattice formation that can be visually observed by hemagglutination assay. Hemaglutinin is involved in the binding of the virus particle to the cell receptor and it also aids in fusion of the viral envelope with the cell membrane to allow the nucleocapsid to enter the cytoplasm of the cell.

The Influenza A virus, like many other eukaryotic viruses, utilizes multiple strategies to ensure the efficient and selective translation of its mRNAs during infection. It encodes mechanisms to down-regulate the interferon-induced, dsRNA-activated protein kinase, PKR, during infection.²⁸ During influenza virus infection, protein synthesis from exogenously introduced cellular genes is subject to host cell shutoff.

Researchers have examined host gene expression patterns associated with influenza types A, B and/or C, including the particularly virulent strains associated with the 1918 Spanish Flu^{8,18} and the 1967 Hong Kong Flu. Using DNA microarray technology, Geiss et al. measured mRNA expression of 13,000+ cellular genes and found that over 300 genes were differentially expressed eight hours after infection.

Comparison of influenza viruses containing mutations in the NS1 protein with the parental wt PR8 virus showed differences in the number and identify of genes re-regulated, with up- or down-regulation observed in one-half to one percent of all cellular genes examined.²⁹

Thus, it is understandable why epidemics and pandemics occur because influenza A virus, which is readily and rapidly transmitted from humans to humans by aerosol, has the capacity to change.⁷ The dramatically virulent strains associated with the 1918-1919 Spanish Flu pandemic and the 1997 Hong Kong outbreak contained changes in the hemaglutinin (HA), which is the major surface protein of the virus. Host cells produce antibodies against the HA protein. The work of Geiss suggests that the NS1 Gene in the infecting virus influences how human lung cells respond to infection immunologically.⁸

Studies of the H5N1/97 influenza virus (1977 Hong Kong virus) found gene expression effects on genes involved with the host immune response. Up-regulation of mRNA for TNFα, interferon beta, RANTES (regulated on activation, normal T cell expressed and secreted), macrophage inflammatory protein (MIP) 1α and 1β, and monocyte chemotactic protein 1 (MCP-1) was much greater after infection with the H5N1/97 viruses 486/97 and 483/97 than after infection with H3N2 or H1N1 viruses. In addition, H5N1/97 virus also differentially up-regulated interferon alpha and interleukins 12, 10, 1β, and 4.

Examples of Systems Biology Linkages Related to Genes Involved with Influenza Infection

Genes that are known to be up- or down-regulated after influenza infection do not work in a vacuum. They are part of a larger system that connects them to other genes, to various chemicals in the environment, and to other types of health conditions.

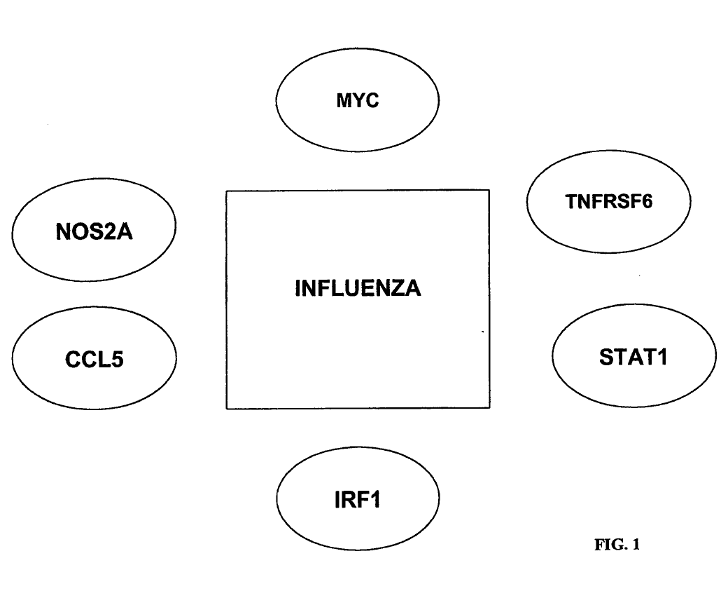

Table 1 lists some of the genes known to be differentially regulated after Influenza A infection. Six of these genes were selected to assess their systems biology relationships. FIGS. 1 to 13 illustrate how genes, small molecules and chemicals, and diseases are interlinked. These figures also demonstrate how the number of interacting components quickly grows.

FIG. 1 applies six of the human genes (ovals) whose gene expression is influenced by influenza A virus (rectangle). See Table 1 for the identity of these and other genes that are differentially regulated by influenza A.

TABLE 1

| | | | Expression | | | | | |
|---|---|---|---|---|---|---|---|---|
| | | Viral | | NS1 | WT | | | |
| Gene ID | Gene Description | Strain | delNS1* | (1-126)* | PR8* | H5N1/97** | H3N2** | H1N1** |
| ABCB2 | ATP-binding cassette, sub-family B, (TAP1) | | Up | up | up | | | |
| BIRC2 | I baculoviral IAP repeat-containing 2 | | Up | up | down | | | |
| BIRC3 | Baculoviral IAP repeat-containing 3 | | Up | up | up | | | |
| GBP1 | Guanylate binding protein 1, IFN-inducible | | Up | up | up | | | |
| IFI16 | IFN-γ-inducible protein 16 | | Up | up | up | | | |
| IFI27 | IFN-α-inducible protein 27 | | Up | up | up | | | |
| IFI41 | IFN-induced protein 41 | | Up | up | up | | | |
| IFI75 | IFN-induced protein 75 | | Up | up | up | | | |
| IFIT1 | IFN-induced protein with tetratricopeptide | | Up | down | up | | | |
| | repeats 1, p56 | | | | | | | |
| IFITM1 | IFN induced transmembrane protein 1 (9-27) | | Up | up | up | | | |
| IFNA | Interferon alpha | | | | | up | | |
| IFNB | Interferon beta | | | | | up | up | up |
| IL10 | Interleukin 10 | | | | | up | | |
| IL12 | Interleukin 12 | | | | | up | | |
| IL1B | Interleukin 1beta | | Up | up | up | up | | |
| IL4 | Interleukin 4 | | | | | up | | |
| IL6ST | IL-6 (IFN, β-2) | | Up | up | up | | | |
| IL8 | IL-8 | | Up | up | down | | | |
| IRF1 | IRF1 IFN regulatory factor 1 | | Up | up | up | | | |
| IRF2 | IFN regulatory factor 2 | | Up | up | up | | | |
| IRF7 | IFN regulatory factor 7 | | Up | up | up | | | |
| ISG15 | IFN-stimulated protein | | Up | up | up | | | |
| ISG20 | IFN stimulated gene | | Up | up | up | | | |
| ISGF3 | IFN-stimulated transcription factor 3-γ, p48, IRF9 | | Up | up | up | | | |
| MCP-1 | Monocyte chemotactic protein 1 | | | | | up | | |
| MIP1A | Macrophage inflammatory protein 1alpha | | | | | up | up | up |
| MIP1B | Macrophage inflammatory protein 1beta | | | | | up | up | up |
| MX1 | Myxovirus (influenza) resistance 1, (MXA) | | Up | up | up | | | |
| MX2 | Myxovirus (influenza) resistance 2, (MXB) | | Up | up | up | | | |
| MYC | V-myc avian myelocytomatosis viral oncogene homolog | | Up | up | up | | | |
| NFKBIA | NF-κB inhibitor, α | | Up | down | up | | | |
| NMI | N-myc (and STAT) interactor | | Up | up | up | | | |
| NOS2A | Nitric oxide synthase 2A | | Up | down | down | | | |
| PRKR | Protein kinase, IFN-inducible double stranded | | Up | up | down | | | |
| | RNA dependent, (PKR) | | | | | | | |
| PTGS2 | Prostaglandin-endoperoxide synthase 2 | | Up | up | down | | | |
| RANTES | RANTES | | | | | up | up | up |
| RANTES | Small inducible cytokine A5 (RANTES) | | Up | up | up | | | |
| RI58 | Retinoic acid- and IFN-inducible protein | | Up | up | up | | | |
| SOD2 | Superoxide dismutase 2, mitochondrial | | Up | up | up | | | |
| SSI-3 | STAT induced STAT inhibitor 3 | | Up | up | up | | | |
| STAF50 | Stimulated trans-acting factor | | Up | up | up | | | |
| STAT1 | Signal transducer and activator of transcription 1, 91 kD | | Up | up | up | | | |
| STAT12 | STAT induced STAT inhibitor-2 | | Down | down | up | | | |
| STAT3 | Signal transducer and activator of transcription 3 | | Up | up | up | | | |
| TGM2 | Transglutaminase2 | | Up | up | up | | | |
| TNFA | Tumor necrosis factor alpha | | | | | up | up | up |
| TNFRSF6 | Tumor necrosis factor receptor superfamily, | | Up | up | down | | | |
| | member 6, (FARS) | | | | | | | |

*Gary K. Geiss, Mahru C. An, Roger E. Bumgarner, Erick Hammersmark, Dawn Cunningham, and Michael G. Katze, Journal of Virology, May 2001, p. 4321-4331, Vol 75 No. 9
**C Y Cheung, L L M Poon, A S Lau, W Luk, Y L Lau, K F Shortridge, S Gordon, Y Guan and J S M Peiris, The Lancet Volume 360, Issue 9348, 7 Dec. 2002, Pages 1831-1837

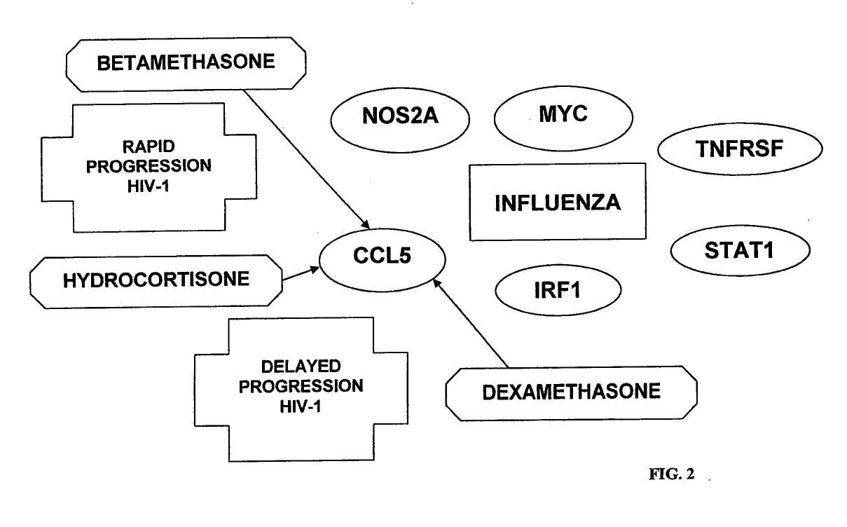

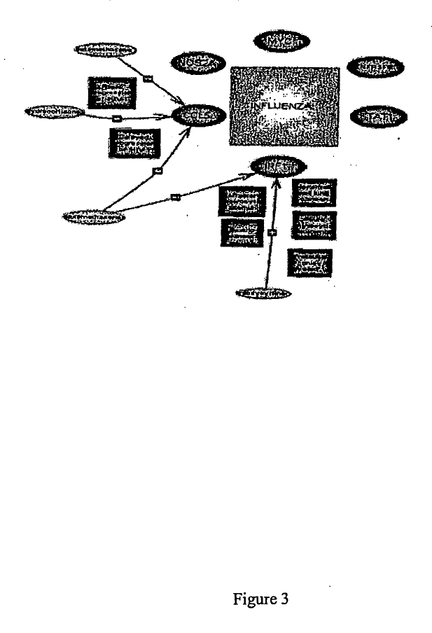

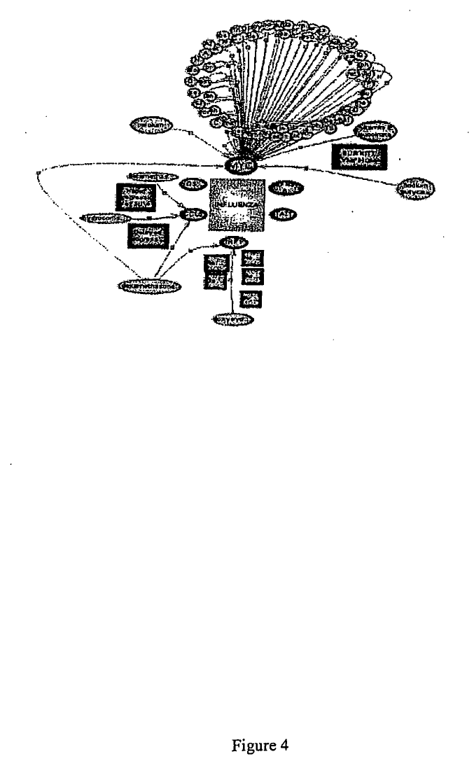

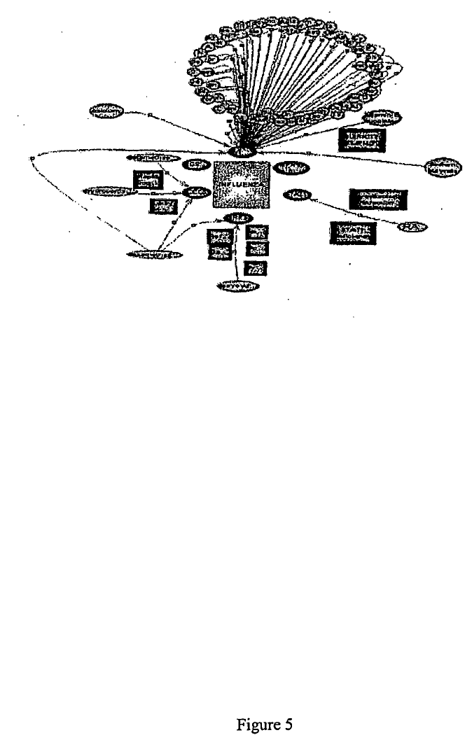

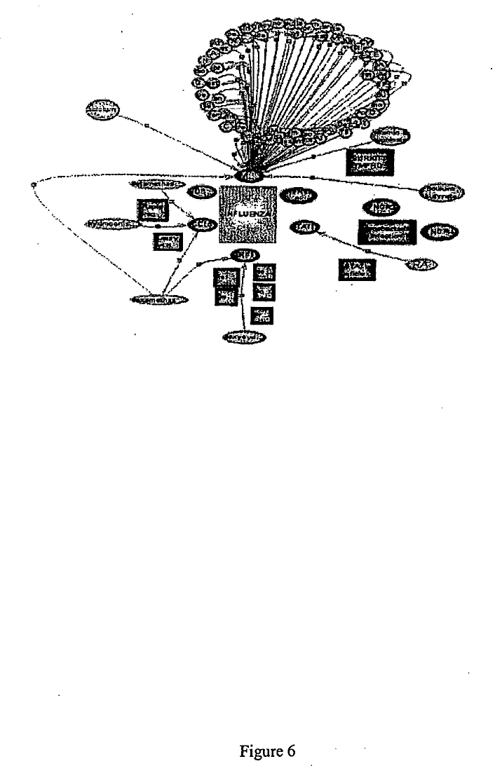

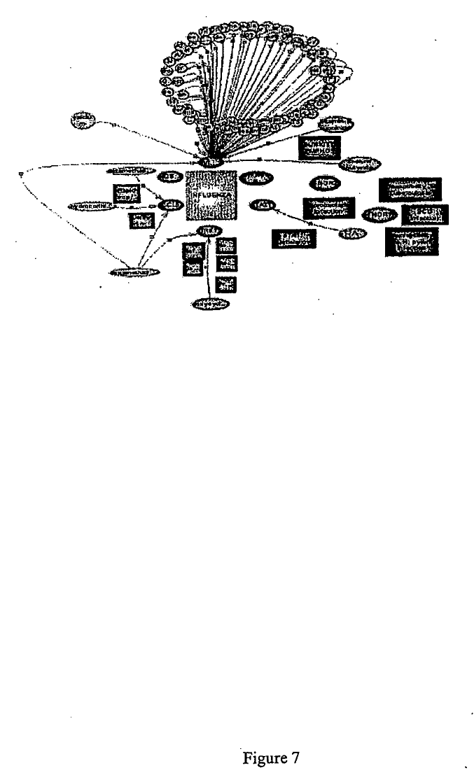

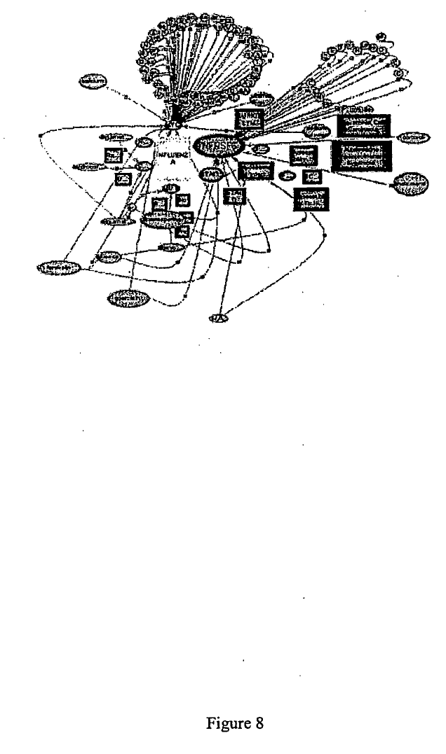

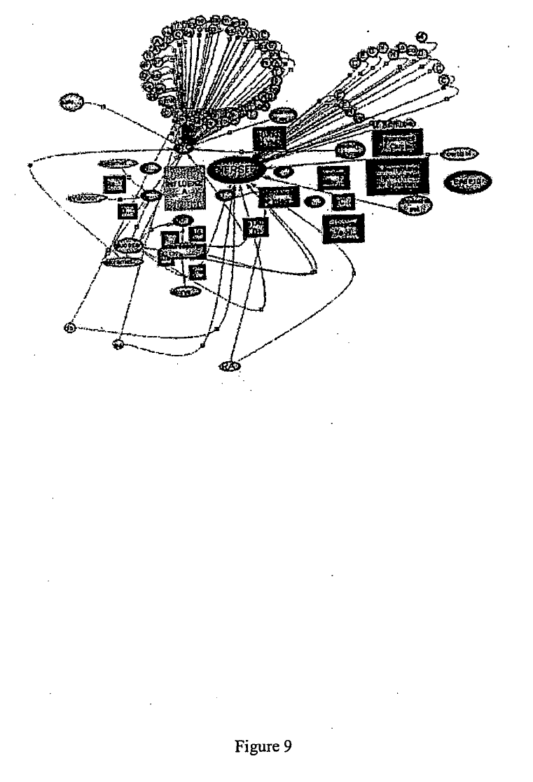

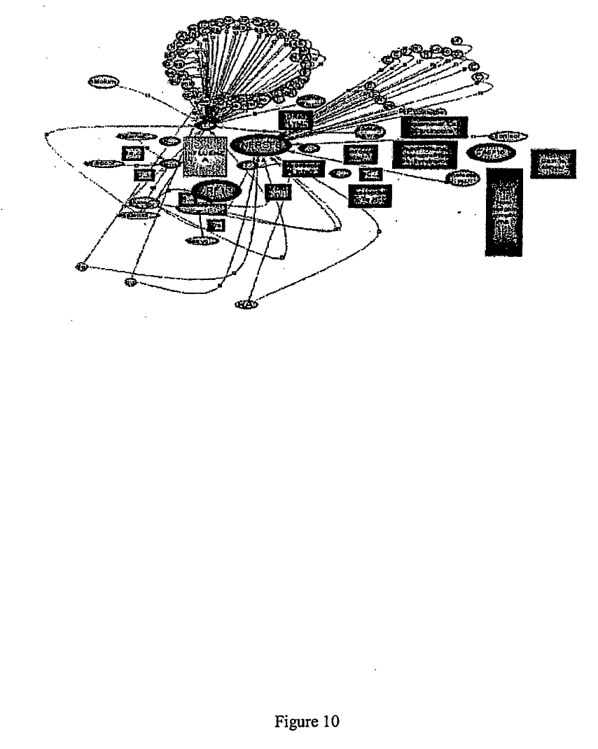

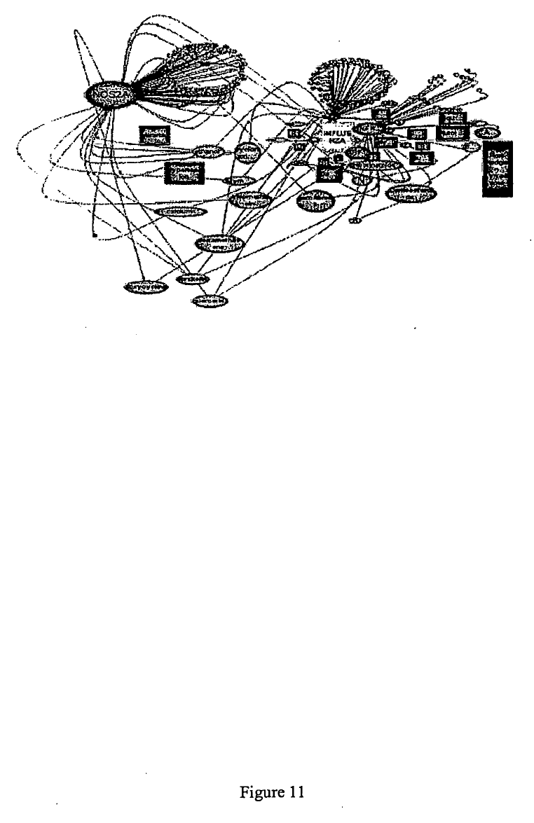

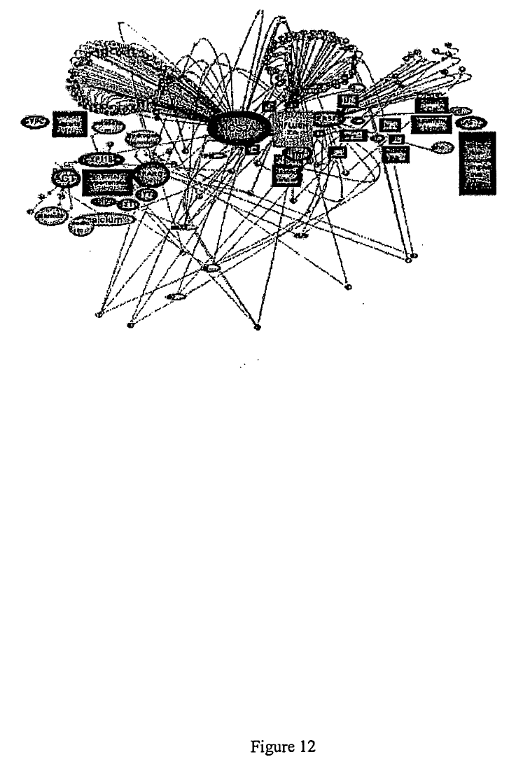

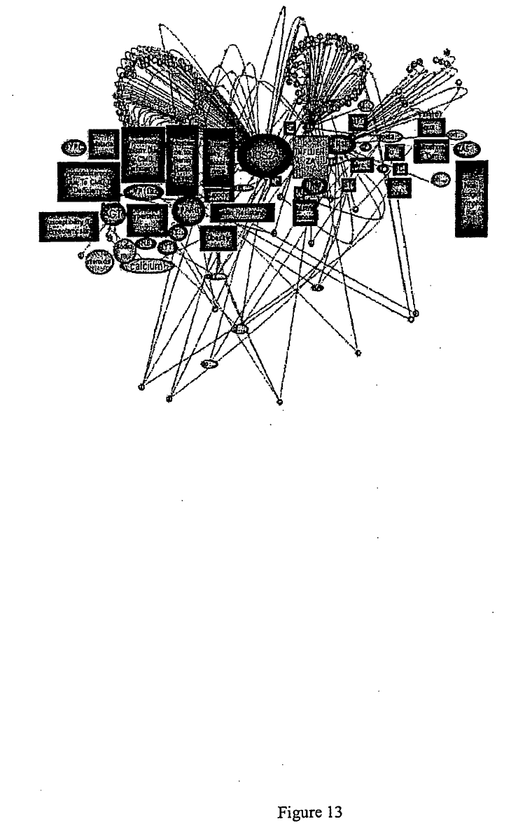

FIG. 2 shows how gene expression of one of the six genes from FIG. 1 (CCL5) is controlled by three small molecules (octagons) and also plays a role in two other health conditions, the rapid and delayed progression of HIV. Expression of CCL5 is also influenced by hydrocortisone, dexamethasone, and betamethasone, and the CCL5 gene has an effect on rapid progression of HIV-1 disease and delayed progression of HIV-1 disease.

One of the small molecules influencing CCL5, dexamethasone, plays a role in another gene: IRF1. IRF1, moreover, is implicated in 6 other health conditions and has at least one other small molecule regulating it. The expression of gene IRF1 may be controlled by doxycycline and dexamethasone. Dexamethasone also controls expression of gene CCL5 as noted in FIG. 2. The IRF1 gene affects refractory macrocytic anemia, gastric cancer, non-small cell lung cancer, acute mylocytic leukemia, and preleukemic myelodysplastic syndrome.

Dexamethasone can regulate the MYC gene. Expression of the MYC gene, moreover, is influenced by over 50 small molecules, including calcium, vitamin E succinate, and sodium butyrate. The MYC gene affects Burkitt's lymphoma.

Retinoic Acid influences the STAT1 gene, which is implicated is mycobacterial infection. Mycobacterium infection, in turn, affects expression in the IFNGR1 and IFNGR2 genes, and the IFNGR1 gene is related to tuberculosis susceptibility. BCG infection, and susceptibility to H. pylori infection.

The interaction of genes and effectors can be complex. Small molecules can influence expression of the TNFRSF6 gene, including cholesterol and cortisol. Other TNFRSF6-influencing small molecules, such as glucose, quercetin, forskolin, and staurosporine help control the MYC gene while retinoic acid influences STAT1. The gene TNFRSF6 affects squamous cell carcinoma and autoimmune lymphoproliferative syndrome.

Multi-dimensional linkages can interact and increase complexity of gene interactions. Increasing numbers of small molecules are identified that influence gene expression. The portfolio of genes involved broadens, because new genes become implicated by sharing common small molecule regulators or by relating to common health outcomes in addition to influenza. The number of health conditions that appear to be indirectly linked to influenza A infection include non-Hodgkin lymphoma somatic, gastric cancer, hypertension, malaria, susceptibility to pre-eclampsia, insulin resistance, lipody, susceptibility to glioblastoma, and obesity. Autoimmune lymphoproliferative syndrome can affect the gene expression of CASP10. CASP10 affects additional health conditions: non-hodgkin lymphoma somatic and gastric cancer. Gastric cancer is a health condition that also affects IRF1. NOS2A may be controlled by many small molecules, a couple of which are cholesterol and cortisol. Some of the small molecules also control the gene TNFRSF6 (gemfibrozil, NO, nicotinamide, corticosteroids, quercetin, forskolin), IRF1 (doxycycline, dexamethasone), MYC (dexamethasone, quercetin, forskolin, indomethacin, silymarin, cycloheximide, ethanol). The gene NOS2A affects hypertension and malaria. Malaria affects the gene expression of GYPC, and hypotension affects the genes, AGT, PPARG, CYP11B2, GNB3, HYT1, and HYT2. AGT affects an additional health condition of susceptibility to preeclampsia. PPARG affects additional health conditions: insulin resistance, lipody, susceptibility to glioblastoma, and obesity. CYP11B2 affects health conditions: hypoaldosteronism due to CMO I deficiency, hypoaldosteronism due to CMO II deficiency, and adolescent aldosterone to renin ratio/ratio raised.

Although the invention has been described with reference to the above examples, it will be understood that modifications and variations are encompassed within the spirit and scope of the invention. Accordingly, the invention is limited only by the following claims.

### REFERENCES

1. Fodor, E., Devenish, L., Engelhardt, O. G., Palese, P., Brownlee, G. G., Garcia-Sastre, A., Rescue of influenza A virus from recombinant DNA, (1999), J. Virol. 73, 9679-9682.
2. Neumann, G., Watanabe, T., Ito, H., Watanabe, S., Goto, H., Gao, P., Hughes, M., Perez, D. R., Donis, R., Hoffmann, E., Hobom, G., Kawaoka, Y., Generation of influenza A viruses entirely from cloned cDNAs, (1999) Proc. Natl. Acad. Sci. U.S.A. 96, 9345-9350.
3. Hatta, M., Gao, P., Halfmann, P., Kawaoka, Y., Molecular basis for high virulence of Hong Kong H5N1 influenza A viruses, (2001) Science 293, 1840-1842.
4. Hatta, M., Neumann, G., Kawaoka, Y., Reverse genetics approach towards understanding pathogenesis of H5N1 Hong Kong influenza A virus infection, (2001) Philos. Trans. R. Soc. Lond. B Biol. Sci. 356, 1841-1843.
5. Hay, A. J., Wolstenholme, A. J., Skehel, J. J., Smith, M. H., The molecular basis of the specific anti-influenza action of amantadine, (1985) EMBO J. 4, 3021-3024.
6. Pinto, L. H., Holsinger, L. J., Lamb, R. A., Influenza virus M2 protein has ion channel activity, (1992) Cell 69, 517-528.
7. Air, G. M., Ghate, A. A., Stray, S. J., Influenza neuraminidase as target for antivirals, (1999) Adv. Virus Res. 54, 375-402.
8. Geiss, G. K., Salvatore, M., Tumpey, T. M., Carter, V. S., Wang, X., Basler, C. F., Taubenberger, J. K., Bumgarner, R. E., Palese, P., Katze, M. G., and Garcia-Sastre, A. (2002) Proc. Natl. Acad. Sci. USA 99, 10736-10741; Cellular transcriptional profiling in influenza A virus-infected lung epithelial cells: The role of nonstructural NS protein in the evasion of the host innate defense and its potential contribution to pandemic influenza
9. Balmer, James E. and Blomhoff, Rune, Gene expression regulation by retinoic acid, (2002) Journal of Lipid Research, Vol. 43, 1773-1808.
10. Linnane A W, Kopsidas G, Zhang C, Yarovaya N, Kovalenko S, Papakostopoulos P, Eastwood H, Graves S, Richardson M., Cellular redox activity of coenzyme Q10: effect of CoQ10 supplementation on human skeletal muscle, (2002) Free Radic Res. April;36(4):445-53.
11. Gohil K. Genomic responses to herbal extracts: lessons from in vitro and in vivo studies with an extract of Ginkgo biloba, Biochem Pharmacol. (2002) September;64(5-6):913-7.
12. Wright, P. F., Webster, R. G., (2001). Orthomyxoviruses. In: Knipe, D. M., Howley, P. M. (Eds.), Fields Virology, 4th ed. Lippincott Williams & Wilkins, Philadelphia, pp. 1533-1579.
13. Krug, R. G.; The potential use of influenza virus as an agent for bioterrorism (2003) Antiviral Research 57, 147-150.
14. Beck, M. A., Nelson, H. K, Shi, Q., Van Dael, P., Schiffrin, E. J, Blum, S., Barclay, D. and Levander, O. A., Selenium deficiency increases the pathology of an influenza virus infection, (2001) FASEB Journal, 15: 1481-1483
15. Nelson, H. K, H. K, Shi, Q., Van Dael, P., Schiffrin, E. J, Blum, S., Barclay, D. Levander, O. A. and Beck, M. A, Host nutritional selenium status as a driving force for influenza virus mutations, (2001) FASEB Journal, 15: 1846-1848
16. Lamb, R. A., Krug, R. M., 2001. Orthomyxoviridae: the viruses and their replication. In: Knipe, D. M., Howley, P. M. (Eds.), Fields Virology, 4th ed. Lippincott Williams & Wilkins, Philadelphia, pp. 1487-1532.
17. Krug, R. G.; The potential use of influenza virus as an agent for bioterrorism (2003) Antiviral Research 57, 147-150.
18. Reid, A. H., Fanning, T. G., Hultin, J. V., and Taubenberger, J. K., Origin and evolution of the 1918 “Spanish” influenza virus hemagglutinen gene, (1999) Proc. Natl. Acad. Sci. USA 99, 1651-1656
19. Reid, A. H., Fanning, T. G., Janczewski, T. A., McCall, S, and Taubenberger, J. K., Characterization of the 1918 “Spanish” influenza virus matrix gene segment, (2002) J. Virology 76, 10717-10723
20. Webster, R. G. (1999) Proc. Natl. Acad. Sci. USA 96, 1164-1166
21. Taubenberger, J. K., Reid, A. H. & Fanning, T. G. (2000) Virology 274, 241-245
22. In Harrison's Principles of Internal Medicine, (1994) at 815; and Harrison's Online, Chapter 190.
23. Geiss, PNAS Aug. 6, 2002, citing Simonsen 1997 ref 1
24. In Harrison's Principles of Internal Medicine, (1994); and Harrison's Online, Chapter 190rrison
25. Lamb, R. A., Krug, R. M., 2001. Orthomyxoviridae: the viruses and their replication. In: Knipe, D. M., Howley, P. M. (Eds.), Fields Virology, 4th ed. Lippincott Williams & Wilkins, Philadelphia, pp. 1487-1532.
26. Wright, P. F., Webster, R. G., 2001. Orthomyxoviruses. In: Knipe, D. M., Howley, P. M. (Eds.), Fields Virology, 4th ed. Lippincott Williams & Wilkins, Philadelphia, pp. 1533-1579.
27. Lamb, R. A. & Krug, R. M. (2001) in Virology, eds. Knipe, D. M. & Howley, P. M. (Lippincott, Philadelphia), pp. 1487-1531.
28. Lee, T. G., Tomita, J., Hovanessian, A. G., and Katze, M. G. (1990) Proc. Natl. Acad.
29. Geiss, G. K., Salvatore, M., Tumpey, T. M., Carter, V. S., Wang, X., Basler, C. F., Taubenberger, J. K., Bumgamer, R. E., Palese, P., Katze, M. G., and Garcia-Sastre, A., Cellular transcriptional profiling in influenza A virus-infected lung epithelial cells: The role of nonstructural NS protein in the evasion of the host innate defense and its potential contribution to pandemic influenza. (2002) Proc. Natl. Acad. Sci. USA 99, 10736-10741

What is claimed is:

1. A method of re-regulating the expression of one or more genes of a host to prevent or mediate viral infection comprising:
i) generating a gene expression pattern for a host;
ii) contacting the host with a battery of one or more agents; and
iii) determining the resulting gene expression pattern of the host of step (ii),
wherein determining comprises relating the pattern of gene expression resulting from agent re-regulation with an inability of the virus to establish infection.

2. The method of claim 1, wherein the resulting pattern is indicative of a host whose gene expression environment makes unavailable the cellular machinery necessary to establish a viral infection.

3. The method of claim 2, wherein the cellular machinery is selected from cell surface molecules, nucleic acids, cytoplasmic molecules, or transcription factors.

4. The method of claim 3, wherein said agent up- and/or down-regulates the expression of cell surface molecules or transcriptional factors.

5. The method of claim 1, wherein said re-regulation comprises modulation of one or more host genes.

6. The method of claim 5, wherein said modulation comprises up and/or down regulation of genes.

7. The method of claim 1, wherein the resulting expression pattern serves as a genomic barrier to infection by a virus.

8. The method of claim 1, wherein said one or more agents are selected from foodstuffs, extracts from foodstuffs, or dietary supplements.

9. The method of claim 1, wherein the host is a cell or a multi-cellular organism.

10. A method of preventing or mediating viral infection in a subject in need thereof comprising administering an agent which re-regulates the gene expression profile of said subject, wherein the resulting gene expression profile of the subject correlates with an inability to support the cellular machinery necessary to establish a viral infection.

11. The method of claim 10, wherein the agent is administered in the form of foodstuffs, extracts from foodstuffs, or dietary supplements.

12. A method of identifying agents which prevent or mediate viral infection by re-regulating the gene expression pattern of a host comprising:
i) generating a gene expression pattern for a host;
ii) contacting the host with at least one or more viruses;
iii) determining the gene expression pattern of the host of step (ii);
iv) contacting the host with a battery of one or more agents; and
v) identifying the one or more agents which re-regulate the gene expression pattern of the host,
wherein said identifying comprises determining that the one or more viruses are unable to establish an infection subsequent to contacting the host with said one or more agents.

13. The method of claim 12, wherein the host is a cell or a multi-cellular organism.

14. A method for targeting one or more common genes that prevent infection from a plurality of viral strain comprising:
i) generating VES patterns for a plurality of non-susceptible hosts, wherein the hosts have been exposed to various strains of viruses;
ii) comparing the patterns of the hosts of step (i);
iii) identifying similar expression profiles for one or more genes within the VES patterns; and
iv) generating a similarity profile based on step (iii),
wherein similar expression profiles between one or more genes are associated with one or more host products that when expressed commonly prevent or mediate infection by said plurality of virus strains.
